(19) United States
(12) Patent Application Publication
(10) Pub. No.: US 2008/0090278 A1
(43) Pub. Date: Apr. 17, 2008

(54) METHOD FOR ENHANCING STABILITY OF A COMPOSITION COMPRISING SOLUBLE GLUCOSE DEHYDROGENASE (GDH)

(75) Inventors: Masao KITABAYASHI, Tsuruga-shi (JP); Yuji TSUJI, Tsuruga-shi (JP); Yoshiaki NISHIYA, Tsuruga-shi (JP); Takahide KISHIMOTO, Tsuruga-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: TOYO BOSEKI KABUSHIKI KAISHA, Osaka-shi (JP)

(21) Appl. No.: 11/931,509

(22) Filed: Oct. 31, 2007

Related U.S. Application Data
(63) Continuation-in-part of application No. 11/694,489, filed on Mar. 30, 2007.
(60) Provisional application No. 60/788,252, filed on Mar. 31, 2006.
(60) Provisional application No. 60/868,276, filed on Dec. 1, 2006.

(51) Int. Cl. C12N 9/96

(52) U.S. Cl. 435/188

(57) ABSTRACT

The present invention relates to a method for enhancing stability of a composition comprising soluble glucose dehydrogenase (GDH). Soluble GDH is preferably FAD-dependent GDH derived from filamentous fungus, and the best effect is observed in FAD-GDH derived from A. oryzae or FAD-GDH derived from A. terreus. According to the invention, in a composition comprising soluble glucose dehydrogenase (GDH), stability of GDH can be enhanced by coexisting the enzyme with one or more compounds selected from amino acids and sugars which are not substrate of the enzyme, thus expected to enhancing a measurement accuracy of glucose.

### TECHNICAL FIELD

The present invention relates to a method for enhancing stability of a composition comprising soluble glucose dehydrogenase (herein also sometimes abbreviated as “GDH”) requiring a coenzyme and the composition using the method. More particularly, the present invention relates to a method for enhancing the stability of a composition comprising soluble glucose dehydrogenase requiring a flavin compound as the coenzyme and the composition using the method.

### BACKGROUND ART

Self-monitoring of blood glucose is important for a patient with diabetes to figure out a usual blood glucose level in the patient and apply it to treatment. An enzyme taking glucose as a substrate is utilized for a sensor used for the self-monitoring of blood glucose. An example of such an enzyme includes, for example, glucose oxidase (EC. 1.1.3.4). Glucose oxidase is advantageous in that it has high specificity for glucose and is excellent in thermal stability, and thus has been used as the enzyme for a blood glucose sensor from a long time ago. Its first publication goes back 40 years ago. In the blood glucose sensor using glucose oxidase, the measurement is performed by transferring electrons produced in a process of oxidizing glucose to convert into D-glucono-δ-lactone to an electrode via a mediator. However, glucose oxidase easily transfers protons produced in the reaction to oxygen, and thus dissolved oxygen affects the measured value, which has been problematic.

In order to avoid such a problem, for example, NAD(P)-dependent glucose dehydrogenase (EC. 1.1.1.47) or pyrrolo-quinoline quinone (herein also described as PQQ)-dependent glucose dehydrogenase (EC. 1.1.5.2; former EC. 1.1.99.17) is used as the enzyme for the blood glucose sensor. They dominates in that they are not affected by dissolved oxygen, but the former NAD(P)-dependent glucose dehydrogenase (herein also sometimes abbreviated as NAD-GDH) has the poor stability and requires the addition of the coenzyme. Meanwhile, the latter PQQ-dependent glucose dehydrogenase (herein also sometimes abbreviated as PQQ-GDH) is inferior in substrate specificity, reacts with other sugars such as maltose and lactose and thus correctness of the measured value is impaired.

In Patent document 1, flavin-binding type glucose dehydrogenase (herein also sometimes abbreviated as FAD-GDH) derived from genus Aspergillus has been disclosed. This enzyme dominates in that this is excellent in substrate specificity and is not affected by the dissolved oxygen. For the thermal stability, it has been described that a residual activity ratio after being treated at 50° C. for 15 minutes is about 89% and this enzyme is excellent in thermal stability.

Patent document 1: WO2004/058958

### DISCLOSURE OF THE INVENTION

It is an object of the present invention to overcome a shortcoming for thermal stability of the publicly known enzyme as described above and provide a composition capable of being used for a practically more advantageous reagent for measuring a blood glucose level.

As described above, for the thermal stability of FAD-GDH described in Patent document 1, it has been reported that the residual activity ratio after being treated at 50° C. for 15 minutes is about 89%.

However, this is a result of an enzyme preparation absolutely obtained by culturing a wild type strain and purifying from the culture. In our study, in the recombinant enzyme from Escherichia coli, it was found that polysaccharide was not added to the enzyme surface and the thermal stability was remarkably reduced.

The thermal stability of FAD-GDH obtained by us from Aspergillus oryzae strain was compared with that of recombinant FAD-GDH (AOGDH) obtained by expressing an FAD-GDH gene from Aspergillus oryzae in Escherichia coli. Consequently, the former kept about 77% of the activity whereas the latter rFAD-GDH had only about 13% of the activity after being treated at 50° C. for 15 minutes.

We also compared the thermal stability of FAD-GDH obtained from Aspergillus terreus strain (NBRC33026) with that of recombinant FAD-GDH (ATGDH) obtained by expressing an FAD-GDH gene from Aspergillus terreus in Escherichia Coli. Consequently, the former kept about 90% of the activity whereas the latter rFAD-GDH had only about 2% of the activity after being treated at 50° C. for 15 minutes.

In the process for producing chips for the blood glucose sensor, a heating and drying treatment is sometimes given. If the recombinant enzyme is used, a large thermal deactivation potentially occurs. Thus, it has been necessary to enhance the thermal stability.

A tactic for enhancing the stability of PQQ-GDH has been reported in Patent document 2, in which the study using PQQ-GDH modified at a gene level has been reported. However, for a procedure to increase the stability without modifying the enzyme, no possibility thereof has been described.

Patent document 2: WO02/072839

A level capable of being heated and dried refers to a state where the residual activity is 20% or more, preferably a state where the residual activity is 40% or more, and more preferably a state where the residual activity is 60% or more, after being treated at 50° C. for 15 minutes.

The present invention comprises the following.

[1] A method for enhancing stability of glucose dehydrogenase (GDH) comprising a step of making one or more compounds selected from amino acids and sugars which are not substrate of the enzyme coexist with the enzyme in a composition comprising soluble GDH requiring a coenzyme, wherein the stability is enhanced compared with a case where the compound is not made coexist.

[2] The method for enhancing the stability according to [1] wherein a final concentration of each compound coexisting in a solution is 0.01% by weight or more and a total concentration of respective compounds is 30% by weight or less.

[3] The method for enhancing the thermal stability according to [1] or [2] characterized in that the compounds to be added are one or more selected from the group consisting of trehalose, mannose, melezitose, sodium gluconate, sodium glucuronate, galactose, methyl-a-D-glucoside, cyclodextrin, a-D-melibiose, sucrose, cellobiose, glycine, alanine, serine, BSA, sodium chloride, sodium sulfate, trisodium citrate, ammonium sulfate, succinic acid, malonic acid, glutaric acid, arabinose, sorbitan, 2-deoxy-D-glucose, xylose, fructose, sodium aspartate, glutamic acid, phenylalanine, proline, lysine hydrochloride, sarcosine and taurine.

[4] The method for enhancing the thermal stability according to any of [1] to [3] characterized in that GDH requiring a flavin compound as the coenzyme is derived from filamentous fungus.

[5] A composition comprising soluble GDH whose thermal stability has been enhanced by the method according to any of [1] to [4].

[6] The composition containing GDH according to [5] characterized in that 20% or more of a GDH activity is left after being treated at 50° C. for 15 minutes compared with the activity in the composition stored at 4° C. in the composition comprising recombinant GDH binding a flavin compound.

[7] The composition containing GDH according to [5] characterized in that 10% or more of a GDH activity is left after being treated at 50° C. for 30 minutes compared with the activity in the composition stored at 4° C. in the composition comprising flavin compound-binding GDH.

[8] A method for measuring a glucose concentration using the composition according to any of [5] to [7].

[9] A glucose sensor comprising the composition according to any of [5] to [7].

[10] A method for producing a composition comprising a step of making one or more compounds selected from amino acids and sugars which are not substrate of glucose dehydrogenase (GDH) coexist with GDH in a composition comprising soluble, coenzyme-binding GDH, wherein thermal stability of GDH has been enhanced compared with a case where the compound is not made coexist.

The enhancement of the stability of the GDH composition according to the present invention enables to reduce thermal deactivation of the enzyme when a glucose measurement reagent, a glucose assay kit and a glucose sensor are produced to reduce the amount of the enzyme to be used or enhance a measurement accuracy. It also enables to provide a reagent for measuring the blood glucose level using the GDH composition excellent in storage stability.

### BEST MODES FOR CARRYING OUT THE INVENTION

GDH is an enzyme which catalyzes the following reaction:

D-Glucose+Electron transport substance (oxidation type)→D-glucono-d-lactone+Electron transport substance (reduction type).

GDH is the enzyme which catalyzes the reaction in which D-glucose is oxidized to generate D-glucono-1,5-lactone, and its origin and structure are not particularly limited.

GDH applicable to the method of the present invention is not particularly limited as long as it is soluble glucose dehydrogenase (GDH).

As a coenzyme, for example, a flavin compound can be taken.

GDH (FAD-binding GDH) applicable to the method of the present invention and taking FAD as the coenzyme is not particularly limited, and includes, for example, those derived from microorganisms which are filamentous fungi belonging to genus Penicillium or Aspergillus belonging to the category of the eukaryotic organisms. These fungal strains are easily available by asking an assignment to the culture collection for respective fungi.

For example, Penicillium lilacinoechinulatum belonging to genus Penicillium has been registered as the deposit numbers NBRC6231 at Biological Resource Center, National Institute of Technology and Evaluation. Aspergillus terreus belonging to genus Aspergillus has been registered as deposit numbers NBRC33026 at Biological Resource Center, National Institute of Technology and Evaluation.

For Aspergillus oryzae, an outline of the procedure to acquire the GDH gene derived from Aspergillus oryzae is as follows.

In order to acquire the GDH gene derived from Aspergillus oryzae, the purification of GDH from the culture supernatant of Aspergillus oryzae and Aspergillus terreus was tried using salting out, chromatography and the like, but it was difficult to yield GDH with high purity (Experiment 1 [1])

Therefore, we had no choice but to give up the cloning utilizing the partial amino acid sequence, which was one of standard methods to acquire the gene.

Thus, we searched GDH-producing microorganisms other than the above microorganisms, and as a result of an extensive study, we found that Penicillium lilacinoechinulatum NBRC6231 produced GDH, and succeeded to yield the purified enzyme with high purity from the culture medium of this fungal strain (Experiment 1 [2]).

Subsequently, we succeeded to determine the partial amino acid sequence using the above enzyme, partially acquired the GDH gene derived from P. lilacinoechinulatum NBRC6231 by PCR based on the determined amino acid sequence and determined its base sequence (1356 bp) (Experiment 1 [3] and [4]).

Finally, based on this base sequence, the GDH gene derived from Aspergillus oryzae was presumed (Experiment 1 [5]) from the published database of Aspergillus oryzae genome, and it was acquired.

<Experiment 1>

Estimation of Glucose Dehydrogenase Gene Derived from Aspergillus oryzae (Hereinafter Also Sometimes Abbreviated as “GDH”)

[1] Acquisition of GDH Derived from Aspergillus oryzae

Aspergillus oryzae obtained from soils and stored as dried microbial cells according to standard methods was used. This is referred to as Aspergillus oryzae TI strain below.

Aspergillus oryzae TI strain was restored by inoculating its dry microbial cells in the potato dextrose agar medium (supplied from Difco) and incubating at 25° C. Fungal threads restored on the plate were collected including the agar, which was then suspended in filtrated sterilized water. In two 10 L jar fermenters 6 L of a production medium (1% malt extract, 1.5% soy bean peptide, 0.1% MgSO₄.7H₂O, 2% glucose, pH 6.5) was prepared and sterilized by autoclave at 120° C. for 15 minutes. After cooling, the above fungal thread suspension was inoculated, and cultured with ventilation and stirring at 30° C. The culture was stopped 64 hours after the start of the culture, and a filtrate containing the GDH activity was collected by removing the fungal threads by filtration. Low molecular substances were removed from the collected supernatant by ultrafiltration (molecular weight 10,000 cut off). Then, ammonium sulfate was added at 60% saturation to perform ammonium sulfate fractionation. The supernatant containing the GDH activity was collected by centrifugation, absorbed to the Octyl-Sepharose column, and eluted with ammonium sulfate having the gradient from 60% saturation to 0% to collect fractions having the GDH activity. The resulting GDH solution was applied onto the G-25 Sepharose column to perform the salting out. Ammonium sulfate was added at 60% saturation thereto. The mixture was absorbed to the Phenyl-Sepharose column and eluted with ammonium sulfate having the gradient from 60% saturation to 0% to collect fractions having the GDH activity. The fraction having the GDH activity was heated at 50° C. for 45 minutes, and then centrifuged to yield the supernatant. The solution obtained from the above steps was made a purified GDH preparation (AOGDH). In the above purification process, 20 mM potassium phosphate buffer (pH 6.5) was used as the buffer. In order to determine the partial amino acid sequence of the AOGDH, the further purification was tried using various procedures such as ion exchange chromatography and gel filtration chromatography, but no purified preparation capable of being subjected to the partial amino acid sequencing could be obtained.

Also, we independently searched and obtained the microorganism belonging to Aspergillus terreus, and likewise tried the purification from its culture supernatant by the salting out and the Octyl-Sepharose, but no purified preparation capable of being subjected to the partial amino acid sequencing could be obtained as was the case with Aspergillus oryzae. Typically, using the purification methods commonly used, it is possible to obtain the protein preparation with high purity detected as a clear single band on SDS-PAGE. However, the GDH preparation at such a level could not be obtained. It was speculated that one of its causes was the sugar chain thought to be bound to the enzyme protein. Therefore, we had no choice but to give up the cloning utilizing the partial amino acid sequence of the protein, which was one of standard methods to acquire the gene.

[2] Acquisition of GDH Derived from Filamentous Fungus Belonging to Genus Penicillium

A purified preparation detected to be nearly uniform on SDS electrophoresis was acquired by using Penicillium lilacinoechinulatum NBRC6231 as the GDH producing fungus derived from the filamentous fungus belonging to genus Penicillium and performing the culture and the purification according to the same procedure as in the case with the above Aspergillus oryzae T1 strain.

[3] Preparation of cDNA

For Penicillium lilacinoechinulatum NBRC6231, according to the above methods, the culture was carried out (but, the culture in the jar fermenter was performed for 24 hours), and the fungal threads were collected by filter paper filtration. The collected fungal threads were immediately frozen in liquid nitrogen, and disrupted using Cool Mill (supplied from Toyobo Co., Ltd.). The total RNA was immediately extracted from disrupted microbial cells using Sepasol RNA I (supplied from Nacalai Tesque) according to the protocol of this kit. mRNA was purified from the resulting total RNA using Origotex-dt30 (supplied from Daiichi Pure Chemicals Co., Ltd.), and RT-PCR with this as the template was performed using ReverTra-Plus™ supplied from Toyobo Co., Ltd. A resulting product was electrophoresed on agarose gel and a portion corresponding to a chain length of 0.5 to 4.0 kb was cut out. cDNA was extracted from a cut out gel fragment using MagExtractor-PCR&Gel Clean Up supplied from Toyobo Co., Ltd. and purified to use as a cDNA sample.

[4] Determination of GDH Gene Partial Sequence

The purified GDH derived from NBRC6231 was dissolved in Tris-HCl buffer (pH 6.8) containing 0.1% SDS and 10% glycerol, and partially digested by adding Glu specific V8 endoprotease at a final concentration of 10 μg/mL thereto and incubating at 37° C. for 16 hours. This sample was electrophoresed on 16% acrylamide gel to separate peptides. Peptide molecules present in this gel were transferred on a PVDF membrane using the buffer for blotting (1.4% glycine, 0.3% Tris and 20% ethanol) by semi-dry method. The peptides transferred onto the PVDF membrane were stained using a CBB staining kit (GelCode Blue Stain Reagent supplied from PIERCE), two band portions of the visualized peptide fragments were cut out and internal amino acid sequences were analyzed using a peptide sequencer. The resulting amino acid sequences were IGGVVDTSLKVYGT (SEQ ID NO:9) and WGGGTKQTVRAGKALGGTST (SEQ ID NO:10). Based on this sequence, degenerate primers containing mixed bases were made, and PCR was performed using the cDNA derived from NBRC6231 as the template. An amplified product was obtained, and was detected as a single band of about 1.4 kb by agarose gel electrophoresis. This band was cut out, and extracted and purified using MagExtractor-PCR&Gel Clean Up supplied from Toyobo Co., Ltd. The purified DNA fragment was TA-cloned using TArget Clone-Plus, and Escherichia coli JM 109 competent cells (Competent High JM109 supplied from Toyobo Co., Ltd.) were transformed with the resulting vector by heat shock. Among transformed clones, for colonies in which an insert had been identified by blue-white determination, the plasmid was extracted and purified using MagExtractor-Plasmid by miniprep, and the base sequence (1356 bp) of the insert was determined using plasmid sequence specific primers.

[5] Estimation of AOGDH Gene

Based on the determined base sequence, the homology was searched on the home page of “NCBI BLAST” (http://www.ncbi.nlm.nih.gov/BLAST/), and the AOGDH gene was estimated from multiple candidate sequences in consideration of the homology to publicly known glucose oxidation enzymes. The homology of the AOGDH estimated from the search to the GDH partial sequence derived from P. lilacinoechinulatum NBRC6231 was 49% at an amino-acid level.

In the GDH applicable to the method of the present invention, as long as the GDH has the glucose dehydrogenase activity, a part of amino acid residues may be deleted or substituted, or other amino acid residues may be added in those exemplified above.

Such a modification can be easily carried out by those skilled in the art using publicly known technologies in the art. For example, various methods for substituting or inserting a base sequence of a gene encoding a protein in order to introduce a site directed mutation into the protein have been described in Sambrook et al., Molecular Cloning; A Laboratory Manual 2nd edition (1989) Cold Spring Harbor Laboratory Press, New York.

For example, a water soluble fraction containing GDH can be obtained by culturing a natural microorganism producing the above GDH or culturing a transformant obtained by inserting a gene encoding the natural GDH directly or after being mutated into an expression vector (many vectors are known in the art, e.g., plasmid) and transforming a host (many hosts are known in the art, e.g., Escherichia coli) with the expression vector, collecting microbial cells from the medium by centrifugation, disrupting the microbial cells by the mechanical method or the enzymatic method using lysozyme and if necessary adding the chelating agent such as EDTA and the surfactant to solubilize. Alternatively, by the use of an appropriate host-vector system, it is possible to secret the expressed GDH directly in the medium.

A GDH-containing solution obtained as the above could be precipitated by concentration under reduced pressure, membrane concentration, salting out treatment using ammonium sulfate or sodium sulfate or fractional precipitation using a hydrophilic organic solvent such as methanol, ethanol or acetone. The treatment with heat and isoelectric focusing treatment are also the effective purification procedures. The purified GDH can also be yielded by performing gel filtration using the absorbing agent or the gel filtration agent absorption chromatography. It is exchange chromatography and affinity chromatography. It is preferable that the purified enzyme preparation is purified to the extent that the enzyme is detected as a single band on electrophoresis (SDS-PAGE).

Before or after the above step, in order to increase the percentage of a holo type GDH relative to the total GDH enzyme protein, the treatment with heat preferably at 25 to 50° C. and more preferably 30 to 45° C. may be performed.

A concentration of GDH in the present invention is not particularly restricted. An appropriate range is different, depending on the properties of the enzyme used. The concentration at which those skilled in the art can actually determine to measure glucose using the enzyme with sufficient reliability is enough.

For example, the concentration of FAD-GDH in the present invention is not particularly restricted, but in the case of the solution, a lower limit is preferably 0.01 U/mL, more preferably 0.1 U/mL and still more preferably 0.2 U/mL. An upper limit, is preferably 5000 U/mL, more preferably 500 U/mL and still more preferably 50 U/mL. The similar concentration is desirable in a powder preparation or a lyophilized product. For the purpose of preparing a powder preparation, it is possible to make the concentration 5000 U/mL or more.

The medium for culturing the microorganism is not particularly limited as long as the microorganism can grow and produce GDH shown in the present invention, but more suitably is preferably one containing carbon sources, inorganic nitrogen sources and/or organic nitrogen sources required for the growth of the microorganism, and more preferably is a liquid medium suitable for ventilation stirring. In the case of the liquid medium, as the carbon, sources, for example, glucose, dextran, soluble starch and sucrose are exemplified, and the nitrogen sources, for example, ammonium salts, nitrates, amino acids, corn steep liquor, peptone, casein, meat extracts, defatted soy beans and potato extracts are exemplified. As desired, other nutrients (e.g., inorganic salts such as calcium chloride, sodium dihydrogen phosphate and magnesium chloride, and vitamins) may be contained.

The culture is performed according to the method known in the art. For example, spores or growing microbial cells of the microorganism are inoculated in the liquid medium containing the above nutrients, and the microbial cells are grown by leaving stand or ventilation stirring, and preferably the microorganism may be cultured by ventilation stirring. A pH value in the culture medium is preferably 5 to 9 and more preferably 6 to 8. A temperature is typically 14 to 42° C. and preferably 20 to 40° C. The culture is continued typically for 14 to 144 hours, but may be terminated when the amount of expressed GDH is maximized in various culture conditions. As the tactic for finding such a time point, the change of GDH activity is monitored by sampling the culture medium and measuring the GDH activity, and the time point when the increase of GDH activity with time is stopped is regarded as a peak of the activity, and the culture may be terminated.

As the method for extracting GDH from the above culture medium, when GDH accumulated in the microbial cells is collected, only the microbial cells are collected by centrifugation or filtration, and resuspended in a solvent, preferably water or buffer. GDH in the microbial cells can be extracted in the solvent by disrupting the resuspended microbial cells by the publicly known method. As the method for disruption, a lytic enzyme can be used, or the method for physically disrupting may be used. The lytic enzyme is not particularly limited as long as it has the capacity to digest the fungal cell wall, and an example of the applicable enzyme includes “lyticase” supplied from Sigma. The method for disrupting physically includes ultrasonic disruption, glass bead disruption and homogenizing disruption. After the disruption, debris can be removed by centrifugation or filtration to yield a GDH crude extraction solution.

As the culture method of the present invention, a solid culture can also be employed. Preferably, the eukaryotic microorganism having a GDH producing capacity of the presented invention is grown on a bran such as wheat under the appropriate control of temperature and humidity. At that time, the culture may be performed by leaving stand, or may be mixed by stirring. GDH is extracted by adding the solvent, preferably the water or the buffer to the culture to dissolve GDH and removing solid matters such as microbial cells and bran.

The GDH can be purified by appropriately combining various separation technologies typically used depending on the fraction in which the GDH activity is detected. The GDH can be purified from the above GDH extraction solution by appropriately selecting the method from publicly known separation methods such as salting out, solvent precipitation, dialysis, ultrafiltration, gel filtration, unmodified PAGE, SDS-PAGE, ion exchange chromatography, hydroxyapatite chromatography, affinity chromatography, reverse phase high performance liquid chromatography and isoelectric focusing electrophoresis.

One mode of the method for enhancing the heat stability of GDH of the present invention comprises a step of making (1) the enzyme coexist with (2) one or more compounds selected from amino acids and sugars which are not the substrate for the enzyme in a composition comprising the enzyme.

Preferable compounds to be added can include one or more selected from the group consisting of trehalose, mannose, melezitose, sodium gluconate, sodium glucuronate, galactose, methyl-a-D-glucoside, cyclodextrin, a-D-melibiose, sucrose, cellobiose, glycine, alanine, serine and BSA.

The concentration of each compound made coexist is not particularly limited. In the case of the solution, the lower limit is preferably 0.001% by weight, more preferably 0.01% and still more preferably 0.1%. In terms of risk to be contaminated with foreign substances, the upper limit is preferably 30% by weight, more preferably 20% and still more preferably 10%. The concentration of the compound described in Examples is represented by % by weight relative to the solvent in the case of the solution, and represented by % by weight relative to the GDH enzyme in a powdered dry matter. For example, in the powdered dry matter, when the stabilizing agent at 60% is added to 40 mg/mL of GDH, 24 mg of the stabilizing agent is added, and at that time, the concentration in the solution is about 2.4%. In an experiment for examining the stabilization in the powder, the GDH in the powder was stabilized within the concentration-range in which the thermal stabilization was observed in the solution. It is easily presumed that the stabilization effect is exerted in the powder in the same concentration range as in the solution.

The concentration of each compound made coexist is not particularly limited. In the solution, the lower limit is preferably 0.01 mM, more preferably 0.1 mM and still more preferably 1 mM. The upper limit is preferably 10 M, more preferably 5 M and still more preferably 1 M.

When the powder or the lyophilized matter is produced, by giving a drying treatment to the composition containing the compound at the similar concentration to in the solution, it is possible to acquire a dry preparation having the same effect as in the solution.

The concentration of the compound described in Examples is a final concentration when stored by coexisting with the GDH enzyme.

The GDH of the present invention can be provided in a liquid form, but can be powderized by lyophilization, vacuum drying or spray drying. At that time, the GDH can be dissolved in the buffer, and further sugars/sugar alcohols, amino acids, proteins and peptides other than the above compounds can be added as excipients or the stabilizing agents. The GDH can be further granulated after being powderized.

The composition of the buffer used for the extraction, purification and powderization of the GDH described above, and a stability test is not particularly limited, could be those having a buffer capacity in the range at pH 5 to 8, and, for example, buffers such as boric acid, Tris hydrochloride and potassium phosphate, and Good's buffers such as BES, Bicine, Bis-Tris, CHES, EPPS, HEPES, HEPPSO, MES, MOPS, MOPSO, PIPES, POPSO, TAPS, TAPSO, TES and Tricine are included. Also the buffers based-on dicarboxylic acid such as phthalic acid, maleic acid and glutaric acid can also be included.

Among them, only one may be applied or tow or more may be used. Furthermore, the buffer may be a composite composition of one or more containing one other than the above.

The concentration of these to be added is not particularly limited as long as it is in the range having the buffering capacity. The upper limit is preferably 100 mM or less and more preferably 50 mM or less. The preferable lower limit is 5 mM or more.

The content of the buffer in the powder or the lyophilized matter is not particularly limited, and the buffer is used in the range of preferably 0.1% (weight ratio) or more and more preferably 0.1 to 80% (weight ratio).

As these, various commercially available reagents can be used.

It is desirable that the various compounds described above are added before making a reagent for measuring the glucose level, a glucose assay kit or a glucose sensor, but they may be added upon measurement. They can also be added in a step solution in each step of extracting, purifying or powderizing GDH.

Enhancement of the thermal stability referred to herein means increasing of a residual ratio (%) of the GDH enzyme kept after giving the treatment with heat at a certain temperature for a certain time period to the composition comprising the GDH enzyme. In the present invention, the activity in the sample stored at 4° C. which is nearly completely kept is made 100%, this is compared with the activity in the GDH solution after giving the treatment with heat at a certain-temperature for a certain time period, and the residual ratio of the enzyme is calculated. When this residual ratio was increased compared with that when the compound had not been added, it was determined that the thermal stability of GDH was enhanced.

Specifically, whether the stability was enhanced or not was determined as follows.

In the method for measuring the activity described in the method for measuring the GDH enzyme activity described later, an activity value (a) of GDH in the solution stored at 4° C. and an activity value (b) of GDH after giving the treatment with heat at a certain temperature for a certain time period were measured, a relative value[(b)/(a)×100] when the activity value (a) was made 100 was calculated. This relative value was made the residual ratio (%). Comparing the presence with the absence of the added compound, when the residual ratio was increased by the addition of the compound, it was determined that the thermal stability was enhanced.

The effect of the present invention becomes remarkable in the system comprising a mediator. The mediator applicable, to the method of the present invention is not particularly limited, and includes the combination of phenazine methosulfate (PMS) with 2,6-dichlorophenolindophenol (DCPIP), the combination of PMS with nitroblue tetrazolium (NBT), DCPIP alone, ferricyanide ion (as the compound, potassium ferricyanide) alone, ferrocene alone and nitrosoaniline alone. Among them, the ferricyanide ion (as the compound, potassium ferricyanide) is preferable.

These mediators are variously different in sensitivity. Thus, the concentration of the mediator to be added is not necessary to be defined uniformly, and generally it is desirable to add at a concentration of 1 mM or more.

These mediators may be added upon measurement or can also be previously contained when producing the reagent for measuring the glucose level, the glucose assay kit or the glucose sensor described later. At that time, the mediator can be added so that it is dissociated to become the ions regardless of a liquid state or a dry state.

In the present invention, it is possible to make various components coexist if necessary. For example, the surfactant and the like may be added.

In the present invention, the glucose level can be measured by the following various methods.

The reagent for measuring the glucose level, the glucose assay kit or the glucose sensor of the present invention can take various forms such as a liquid (aqueous solution, suspension), a powderized one by vacuum drying or spray drying and a lyophilized one. A drying method is not particularly limited, and could be performed in accordance with standard methods. The composition comprising the enzyme of the present invention is not limited to the lyophilized matter, and may be in the solution state obtained by re-dissolving the dry matter.

In the present invention, the glucose level can be measured by the following various methods.

Reagent for Measuring Glucose Level

The reagent for measuring the glucose level of the present invention typically includes the reagents such as GDH, buffer and mediator required for the measurement, glucose standard solutions for making a calibration curve and instructions for the use. The kit of the present invention can provide as the lyophilized reagent or as the solution in an appropriate storage solution. Preferably, the GDH of the present invention is provided as a holoenzyme, but can be provided as an apoenzyme and converted into the holoenzyme in use.

Glucose Assay Kit

The present invention is characterized by the glucose assay kit containing GDH according to the present invention. The glucose assay kit of the present invention contains GDH according to the present invention in a sufficient amount for at least one assay. Typically, the kit includes the buffer, the mediator, essential for the assay in addition to GDH, glucose standard solutions for making the calibration curve and instructions for the use. The GDH according to the present invention can be provided in various forms, for example, as the lyophilized reagent or as the solution in the appropriate storage solution. Preferably, the GDH of the present invention is provided as the holoenzyme, but can be provided as the apoenzyme and converted into the holoenzyme in use.

Glucose Sensor

The present invention is also characterized by the glucose sensor using the GDH according to the present invention. “As an” electrode, a carbon electrode, a gold electrode, a platinum electrode and the like are used, and the enzyme of the present invention is immobilized on this electrode. As the method for immobilization, the method of using a crosslinking reagent, the method of enfolding in a polymer matrix, the method of covering with a dialysis membrane, photo-crosslinkable polymers, conductive polymers and redox polymers are available. Alternatively, the GDH together with the mediator may be fixed in the polymer or absorbed/fixed on the electrode. Also, the combination thereof may be used. Preferably, the GDH of the present invention is immobilized on the electrode as the holoenzyme, or it is possible to immobilize as the apoenzyme and supply the coenzyme as another layer or in the solution. Typically, the GDH of the present invention is immobilized on the carbon electrode using glutaraldehyde, and subsequently glutaraldehyde is blocked by treating with the reagent having the amine group.

The glucose concentration can be measured as follows. The buffer is placed in a cell at constant temperature, the mediator is added and the temperature is kept constant. As an action electrode, the electrode on which GDH of the present invention has been immobilized is used, and a counter electrode (e.g., platinum electrode) and a reference electrode (e.g., Ag/AgCl electrode) are used. A certain voltage is applied to the carbon electrode and the current becomes constant, and subsequently the increase of the current is measured by adding the sample containing glucose. According to the calibration curve made from the glucose solutions at standard concentrations, the glucose concentration in the sample can be calculated.

### EXAMPLES

The present invention will be more specifically described below by Examples.

### Test Example 1

### Method for Measuring FAD-Dependent GDH Activity

In the present invention, the activity of FAD-dependent GDH is measured as follows.

<Reagents>

50 mM PIPES buffer pH 6.5 (containing 0.1% Triton X-100)

163 mM PMS solution

6.8 mM 2,6-dichlorophenol-indophenol (DCPIP) solution

1 M D-glucose solution

The reaction-reagent is made by mixing 15.6 mL of the PIPES buffer, 0.2 mL of the DCPIP solution and 4 mL of the D-glucose solution.

<Measurement Condition>

The reaction reagent (3 mL) is preliminarily heated at 37° C. for 5 minutes. The GDH solution (0.1 mL) is added and gently mixed, subsequently the change of absorbance at 600 nm is recorded for 5 minutes using the spectrophotometer controlled to 37° C. using water as the control, and the change of absorbance per one minute (ΔOD_{TEST}) is calculated from the linear portion of the record. The solvent in which GDH will be dissolved in place of the blinded GDH solution is added to the reagent mixture, and the change of absorbance (ΔOD_{BLANK}) per one minute is measured. The GDH activity is calculated from these values according to the following formula. One unit (U) in the GDH activity is defined as the amount of the enzyme which reduces 1 μM DCPIP for one minute in the presence of 200 mM D-glucose.
Activity (U/mL)=[−(ΔOD_{TEST}−ΔOD_{BLANK})×3.0×dilution scale]/(16.3×0.1×1.0)
In the above formula, 3.0 represents a liquid amount (mL) of the reaction reagent+the enzyme solution, 16.3 represents a millimolar molecular absorbance coefficient (cm²/μmol) in the condition for measuring this enzyme, 0.1 represents the liquid amount of the enzyme solution (mL) and 1.0 represents a light path length (cm) of the cell.

### Example 1

### Preparation of Recombinant Glucose Dehydrogenase Preparation Derived from Filamentous Fungus

mRNA was prepared from microbial cells of Aspergillus oryzae TI strain (obtained from soils and stored as L-dried microbial cells according to standard methods. Hereinafter, this is referred to as Aspergillus oryzae TI strain.) and Aspergillus terreus NBRC33026 strain, and cDNA was synthesized. Four oligo DNA shown in SEQ ID NOS:3 and 4 and SEQ ID NOS:7 and 8 were synthesized. Using each, cDNA prepared from each mRNA as the template, GDH genes derived from Aspergillus oryzae and Aspergillus terreus, were amplified using KOD-Plus (supplied from Toyobo Co., Ltd). The resulting DNA fragments were treated with the restriction enzymes NdeI and BamHI, and inserted into NdeI-BamHI sites in pBluescript (the NdeI site had been introduced to match a NdeI recognition sequence ATG to a translation initiation codon ATG of LacZ) to construct two recombinant plasmids (pAOGDH, pATGDH). These recombinant plasmids were introduced into Competent High DH5α (supplied from Toyobo Co., Ltd.). The plasmids were extracted according to the standard method, and the base sequences of the AOGDH gene and the ATGDH gene were determined (SEQ ID NOs:1 and 5). The amino acid sequence deduced from the DNA sequence was composed of 593 amino acid residues (SEQ ID NO:2) in Aspergillus oryzae and 568 amino acid residues (SEQ ID NO:6) in Aspergillus terreus. By the same techniques, the transformant transformed with the recombinant plasmid (pPIGDH) containing the GDH gene (PIGDH) derived from Penicillium italicum was acquired.

These transformants were cultured in the TB medium (2.4% yeast extract, 1.2% polypeptone, 1.25% dipotassium monohydrogen phosphate, 0.23% monopotassium dihydrogen phosphate, 0.4% glycerol, 50 μg/mL of sodium ampicillin, pH 7.0), using the 10 L jar fermenter at 25° C. at ventilation, amount of 2 L/minute and at a stirring rotation speed of 170 rpm for 48 hours.

The cultured microbial cells were collected by centrifugation, suspended in 50 mM phosphate buffer (pH 5.5) so that the microbial-cell turbidity at 660 nm was about 50, and disrupted with a homogenizer at a pressure of 65 MPa. The nucleic acid was precipitated by adding polyethyleneimine at a final concentration of 9% to the supernatant obtained by centrifuging the disrupted solution, and the supernatant was obtained by centrifugation. Ammonium sulfate in, saturated amount was dissolved in this to precipitate an objective protein, and the precipitate collected by centrifugation was re-dissolved in 50 mM phosphate buffer (pH 5.5). Gel filtration using the G-25 Sepharose column and hydrophobic chromatography using the Octyl-Sepharose column and the Phenyl-Sepharose column (a peak fraction was extracted by eluting with ammonium sulfate with concentration gradient from 25% saturation to 0%) were carried out, and further ammonium sulfate was removed by gel filtration using the G-25 Sepharose column to prepare a recombinant GDH preparation.

### Example 2

### Preparation of FAD-GDH Derived from Wild Type Filamentous Fungi

Using Aspergillus terreus NBRC33026 strain and Aspergillus oryzae TI strain as FAD-dependent GDH-producing fungi derived from the wild type filamentous fungi, each lyophilized fungus was inoculated on the potato dextrose agar medium (supplied from Difco) and incubated at 25° C. to restore. Fungal threads restored on the plate were collected including the agar, which was then suspended in filtrated sterilized water. In two 10 L jar fermenters, 6 L of the production medium (1% malt extract, 1.5% soy bean peptide, 0.1% MgSO₄.7H₂O, 2% glucose, pH 6.5) was prepared and sterilized by autoclave at 120° C. for 15 minutes. Then, the above-fungal thread suspension was added thereto, and the culture was started. The culture was performed under the condition of a temperature at 30° C., a ventilation amount at 2 L/minute and a stirring frequency at 380 rpm. The culture was stopped 64 hours after the start of the culture, and microbial cells from each fungal strain were collected on the filter paper by aspiration filtration using Nutsche filter. The culture medium (5 L) was concentrated to 1/10 amount using a hollow fiber module for ultrafiltration with molecular weight 10,000 cut off, and ammonium sulfate was added to and dissolved in each concentrated solution so that the final concentration was 60% saturation (456 g/L). Subsequently, the mixture was centrifuged at 8000 rpm for 15 minutes using the high speed cooling centrifuge supplied from Hitachi Ltd. to precipitate the cell debris. Then, the supernatant was absorbed to the Octyl Sepharose column, and fractions having the GDH activity were collected by eluting with the gradient of ammonium sulfate from 0.6 to 0.0 saturation. Salting out was performed by applying the resulting GDH solution onto the G-25 Sepharose column for gel filtration and collecting protein fractions. Ammonium sulfate corresponding to 0.6 saturation was added to the solution after the salting out. This mixture was absorbed to the Phenyl Sepharose column, and fractions having the GDH activity were collected by eluting with the gradient of ammonium sulfate from 0.6 to 0.0 saturation. The resulting GDH solution was applied to the gel filtration using the G25 Sepharose column to collect the protein fraction. The acquired purified enzyme was used as the preparation for evaluating FAD-dependent GDH.

The mediator used for the composition for measuring the glucose level, the glucose assay kit, the glucose sensor or the method for measuring the glucose level is not particularly limited, and preferably 2,6-dichlorophenol-indophenol (abbreviated as DCPIP) and ferrocene or derivatives thereof (e.g., potassium ferricyanide, phenazine methosulfate) could be used. As these mediators, commercially available products can be obtained.

### Example 3

### Study on FAD-GDH Thermal Stabilization Effect of Various Stabilizing Agents Using Glucose Measurement System

The study was performed in accordance with the method for measuring the FAD-GDH activity in Test Example 1 described above.

First, 50 mL of an enzyme solution obtained by dissolving the recombinant FAD-GDH (rAO-FAD-GDH)-derived from Aspergillus oryzae obtained in Example 1 at about 2 U/mL in an enzyme dilution solution (50 mM potassium phosphate buffer, pH 5.5, 0.1% Triton X-100) was prepared. Two tubes in which each stabilizing agent described in Table 1 had been added at each final concentration to 0.9 mL of this enzyme solution to make the total volume 1.0 mL were prepared. As the control, two tubes in which 0.1 mL of distilled water had been added in place of each compound were prepared.

In two tubes, one tube was stored at −4° C. and another tube was treated at 50° C. for 15 minutes. Then, the FAD-GDH activity in each tube was measured. The enzyme activity in the tube stored at 4° C. was made 100, and comparing with it, the activity value after being treated at 50° C. for 15 minutes was calculated as the residual activity ratio (%).

As a result of these experiments, it was revealed that the thermal stability of FAD-GDH was increased by adding sugars and certain type amino acids which were not the substrates of FAD-GDH (Table 1). The higher thermal stabilization effect was observed in the sugars than the amino acids. Among them, the high effect was observed in trehalose, mannose, melezitose, sodium gluconate, sodium glucuronate, galactose, methyl-a-D-glycoside, a-D-melibiose, sucrose, glycine, alanine, serine, sodium chloride, sodium sulfate, trisodium citrate, ammonium sulfate, succinic acid, malonic acid, glutaric acid, arabinose, sorbitan, 2-deoxy-D-glucose, xylose, fructose, sodium aspartate, glutamic acid, phenylalanine, proline, lysine hydrochloride, sarcosine and taurine. Among them, the high effect was observed in trehalose, mannose, sodium gluconate, galactose, methyl-a-D-glucoside and a-D-melibiose.

TABLE 1

| | Final | | Residual activity ratio |
|---|---|---|---|
| | concen- | | (%) after treatment at |
| | tration | Stabilizer | 50° C., 15 min |
| | 1% | Control(no stabilizer) | 19.3 |
| Sugars | 1% | D-(−)-arabinose | 23.0 |
| | 1% | 1,4-sorbitane | 26.7 |
| | 1% | 2-deoxy-D-glucose | 69.6 |
| | 1% | D-(+)-xylose | 68.5 |
| | 1% | D-(+)-trehalosedihydrate | 64.7 |
| | 1% | D-(+)-mannose | 65.3 |
| | 1% | D-(+)-melezitose | 44.7 |
| | 1% | D-(−)-fructose | 25.9 |
| | 1% | sodium gluconate | 33.5 |
| | 1% | D-sodium glucuronate | 56.2 |
| | 1% | D-a-galacturonic acid | 0.0 |
| | 1% | Inulin | 24.7 |
| | 1% | Galactose | 60.6 |
| | 1% | Glucono-1,5-lactone | 0.0 |
| | 1% | Methyl-a-D-glucoside | 53.6 |
| | 1% | a-cyclodextrin | 44.1 |
| | 1% | a-D-(+)-melibiose | 59.0 |
| | 1% | Sucrose | 44.1 |
| Amino | 1% | Sodium L-aspartate | 23.0 |
| acids | 1% | L-aspartic acid | 12.2 |
| | 0.20% | L-asparagine | 22.8 |
| | 1% | Glycine | 26.4 |
| | 0.10% | D-glutamic acid | 23.2 |
| | 0.10% | D-phenylalanine | 23.6 |
| | 1% | D-proline | 24.8 |
| | 1% | D-a-alanine | 27.1 |
| | 0.25% | DL-isoleucine | 23.4 |
| | 0.25% | L-glutamine | 22.6 |
| | 1% | L-(−)-proline | 25.2 |
| | 1% | L-arginine | 1.1 |
| | 1% | L-serine | 27.0 |
| | 0.10% | L-triptophan | 24.3 |
| | 0.50% | L-valine | 25.5 |
| | 0.25% | L-histidine | 11.4 |
| | 0.20% | L-phenylalanine | 22.3 |
| | 1% | L-lysine hydrochloride | 23.4 |
| | 0.10% | L-leucine | 22.2 |
| | 1% | Sarcosine | 25.2 |
| | 1% | Taurine | 23.8 |

### Example 4

### Study on Effective Concentration of Trehalose for FAD-GDH Thermal Stabilization Effect

Subsequently, concerning trehalose exhibiting the high thermal stabilization effect, the effective concentration at which its effect was exerted was examined. The method was in accordance with Example 3 described above. As a result, as the concentration of added trehalose was increased, the effect tended to increase. It was revealed that even when trehalose was added at a final concentration of 0.01%, the stabilization effect was exerted (Table 2).

TABLE 2

| | GDH Residual activity ratio |
|---|---|
| Final concentration of | (%) after treatment at 50° C., |
| stabilizer | 30 min |
| Control (no stabilizer) | 3.9 |
| 0.01% trehalose | 11.2 |
| 0.1% trehalose | 20.4 |
| 1% trehalose | 42.3 |

### Example 5

### Study on Other Synergistic Effects

In accordance with Example 3 above for the basic methods, whether the synergistic effect was observed in the other stabilizing agents or not was examined by combining them. As a result, the obvious thermal stabilization effect was confirmed in the combination of serine and BSA (Table 3), the combination (Table 4) of trehalose and mannose, trehalose and glycine, or mannose and glycine compared with a single use.

TABLE 3

| | | GDH Residual |
|---|---|---|
| Stabilizer | GDH Residual activity | activity ratio (%) |
| Final | ratio (%) after treatment | after treatment at |
| concentration | at 50° C., 15 min | 50° C., 15 min |
| Control (no | 13 | 0.5 |
| additives) | | |
| 2.5% BSA | 20 | 3.5 |
| 10% serine | 33 | 11.5 |
| 10% serine + 2.5% BSA | 51 | 25.4 |

TABLE 4

| | GDH Residual activity ratio |
|---|---|
| stabilizer | (%) after treatment at 50° C., |
| final concentration | 30 min |
| control (no additives) | 3.50 |
| 1% trehalose | 39.10 |
| 1% mannose | 47.78 |
| 1% glycine | 4.84 |
| 1% trehalose + 1% mannose | 49.54 |
| 1% trehalose + 1% glycine | 51.93 |
| 1% mannose + 1% glycine | 63.41 |

### Example 6

### Study on Effective Concentrations of Trehalose and Glycine or Mannose and Glycine

Concerning the combination of trehalose and glycine or glycine and mannose which had exhibited the high thermal stabilization effect in Example 5, the effective concentration at which their effect was exerted was examined. The methods were in accordance with Example 3 above. As a result, also in these compounds, as the concentration of the added compound was increased, the effect tended to increase. Even when the compound was added at a final concentration of 0.01%, around 20% residual activity was observed after being treated at 50° C. for 30 minutes, and nearly double stabilization effect was observed compared with the case of using 0.01% trehalose alone (Table 5).

TABLE 5

| | |
|---|---|
| stabilizer | GDH Residual activity ratio (%) |
| final | after treatment at 50° C., 30 min |

| concentration | trehalose + glycine | mannose + glycine |
|---|---|---|
| Each 2% | 63.1 | 71.3 |
| Each 1% | 53.7 | 63.8 |
| Each 0.1% | 29.4 | 44.7 |
| Each 0.01% | 18.7 | 22.9 |

| | |
|---|---|
| no additives | 3.5 |

### Example 7

### Study on Thermal Stabilization Effect of Stabilizing Agent on Various FAD-GDH

In accordance with Example 3 above for the basic methods, the thermal stabilization effect of the stabilizing agent on various FAD-GDH was examined. As the stabilizing agent, a mixed composition of 4% sodium D-glucuronate and 4% glycine was used. As a result, it was demonstrated that the effect of the stabilizing agent was observed regardless of GDH derived from the wild type and recombinant GDH (Table 6).

TABLE 6

| | GDH Residual activity ratio (%) | |
|---|---|---|
| | after treatment at 50° C., 30 min | |
| | stabilizer (−) | Stabilizer (+) |
| A. terreus subspecies wild | 70.0 | 89.2 |
| type GDH | | |
| Recombinant GDH from P. italicum | 2.7 | 92.9 |
| Wild type GDH from A. oryzae | 73.2 | 94.2 |
| Recombinant GDH from A. oryzae | 2.9 | 71.8 |

### Example 8

### Study on Storage Stability Using Glucose Measurement System

The study was performed using the recombinant FAD-GDH preparation (rAO-FAD-GDH) derived from Aspergillus oryzae obtained in Example 1 in accordance with the method for measuring the FAD-GDH activity in Test Example 1 above. The amount of the protein which occupied in the FAD-GDH enzyme solution was measured, and 1 mL of the solution in which the stabilizing agent corresponding to 60% or 30% relative to this had been dissolved therein was prepared. For example, when BSA corresponding to 60% was added to the enzyme solution containing 10 mg of FAD-GDH, 6 mg of BSA was dissolved.

Several vials in which 0.2 mL of the enzyme solution containing each stabilizing agent had been correctly dispensed were prepared. As the control, the vials in which the stabilizing agent had not been added was prepared. The prepared vials were subjected to vacuum freeze-dry (FDR) to completely evaporate the water. Then, only two samples in which the same stabilizing agent had been added were subjected to the measurement of the activity. Meanwhile, the remaining vials were treated at 25° C. at a humidity of 70% for several hours, and then stored at 37° C. for one week. Subsequently, the residual activity was measured. The residual activity ratio (%) was calculated by making the average activity immediately after FDR 100% and measuring the average activity of the samples after storing at 37° C. When the higher the residual activity ratio was, this was determined to further enhance the stability.

As a result, BSA, serine or trehalose alone was observed to enhance the stability of the powdered enzyme, but the further storage stabilization effect was observed by combining BSA and serine. Due to the limited amount of the enzyme preparations, only a few combinations were analyzed, but the compound which had exhibited the thermal stabilization effect seems to likewise have the storage stabilization effect (Tables 7 and 8).

TABLE 7

| | GDH activity (kU/vial) | | GDH activity |
|---|---|---|---|
| | Immediately after | After 1 week | Residual ratio |
| Stabilizer | pulverization | at 37° C. | (%) |
| Control (no | 18.3 | 1.6 | 8.9 |
| additives) | | | |
| 60% BSA | 16.7 | 4.3 | 25.9 |
| 60% serine | 19.8 | 8.2 | 41.3 |
| 60% BSA × 60% | 19.5 | 14.0 | 71.6 |
| serine | | | |

* After treatment at 70% humidity, 25° C., 24 h, and then allowed to stand at 37° C. for 1 week.

TABLE 8

| | GDH activity (U/vial) | | | GDH |
|---|---|---|---|---|
| | Immediately | Immediately | | activity |
| | before | after | At 37° C. | Residual |
| Stabilizer | pulverization | pulverization | for 1 week | ratio (%) |
| Control | 403 | 352 | 6 | 1.6 |
| (no dditives) | | | | |
| 60% trehalose | 397 | 401 | 195 | 48.6 |

* After treatment at 70% humidity, 25° C., 7 h, and then allowed to stand at 37° C. for 1 week.

### Example 9

### Study on FAD-GDH Thermal Stabilization Effect of Various Stabilizing Agents Using Glucose Measurement System

The study was performed in accordance with the method for measuring the FAD-GDH activity in Test Example 1 above.

First, 50 mL of an enzyme solution obtained by dissolving the recombinant FAD-GDH (rAT-FAD-GDH) derived from Aspergillus terreus obtained in Example 1 at about 2 U/mL in an enzyme dilution solution (50 mM potassium phosphate buffer, pH 5.5, 0.1% Triton X-100) was prepared. Two tubes in which each stabilizing agent described in Table 1 had been added at each final concentration to 0.9 mL of this enzyme solution to make the total volume 1.0 mL were prepared. As the control, two tubes in which 0.1 mL of distilled water had been added in place of each compound were prepared.

In two tubes, one tube was stored at 4° C. and another tube was treated at 50° C. for 15 minutes. Then, the FAD-GDH activity in each tube was measured. The enzyme activity in the tube stored at 4° C. was made 100, and comparing with it, the activity value after being treated at 50° C. for 15 minutes was calculated as the residual activity ratio (%).

As a result of these experiments, it was revealed that the thermal stability of FAD-GDH was increased by adding sugars and certain type amino acids which were not the substrates of FAD-GDH (Tables 9 and 10).

The higher thermal stabilization effect was observed in the sugars than the amino acids. Among them, the high effect was observed in trehalose, mannose, melezitose, sodium gluconate, sodium glucuronate, galactose, methyl-a-D-glycoside, a-D-melibiose, sucrose, glycine, alanine, serine, sodium chloride, sodium sulfate, trisodium citrate, ammonium sulfate, succinic acid, malonic acid, glutaric acid, arabinose, sorbitan, 2-deoxy-D-glucose, xylose, fructose, sodium-aspartate, glutamic acid, phenylalanine, proline, lysine hydrochloride, sarcosine and taurine.

TABLE 9

| | | | GLD activity | | Residual |
|---|---|---|---|---|---|
| | | | value (U/ml) | | activity |
| | Compounds | | | 50° C., | ratio |
| Buffer | added | pH | 4° C. | 15 min | (%) |
| 50 mM K-PB | No additives | 6.01 | 0.86 | 0.02 | 2.0 |
| | No additives | 6.51 | 1.25 | 0.01 | 0.7 |
| | Sodium chloride | 5.44 | 1.01 | 0.17 | 16.6 |
| | Sodium sulfate | 5.51 | 1.05 | 0.24 | 23.0 |
| | Trisodium | 6.53 | 1.22 | 0.20 | 16.2 |
| | citrate | | | | |
| | Ammonium | 5.42 | 1.03 | 0.16 | 15.3 |
| | sulfate | | | | |
| | Succinic acid | 6.00 | 1.43 | 0.28 | 19.6 |
| | Malonic acid | 6.07 | 1.43 | 0.21 | 14.9 |
| | Glutaric acid | 5.98 | 1.43 | 0.31 | 21.7 |
| 50 mM Succinic | | 4.98 | 0.98 | 0.02 | 1.6 |
| acid buffer | | | | | |
| | Sodium chloride | 4.66 | 1.01 | 0.06 | 5.4 |
| | Sodium sulfate | 4.73 | 1.09 | 0.11 | 10.5 |
| | Trisodium | 6.29 | 1.43 | 0.56 | 38.9 |
| | citrate | | | | |
| | Ammonium | 4.64 | 1.03 | 0.06 | 5.8 |
| | sulfate | | | | |

TABLE 10

| | | GLD activity | | |
|---|---|---|---|---|
| | | value (U/ml) | | Residual |
| | | | 50° C., | activity |
| Buffer | | 4° C. | 15 min | ratio (%) |
| 50 mM K-PB | Control (no stabilizer) | 3,519 | 0.054 | 1.5 |
| | D-(−)-arabinose | 2,794 | 0.069 | 2.5 |
| | 1,4-sorbitane | 2,931 | 0.072 | 2.5 |
| | 2-deoxy-D-glucose | 2,795 | 1.032 | 36.9 |
| | D-(+)-xylose | 2,796 | 0.516 | 18.5 |
| | D-(+)-trehalosedihydrate | 2,731 | 0.431 | 15.8 |
| | D-(+)-mannose | 2,713 | 0.448 | 16.5 |
| | D-(+)-melezitose | 2,787 | 0.193 | 6.9 |
| | D-(−)-fructose | 2,745 | 0.075 | 2.7 |
| | sodium gluconate | 2,857 | 0.111 | 3.9 |
| | D-sodium glucuronate | 2,790 | 0.279 | 10.0 |
| | D-a-galacturonic acid | 1,644 | 0.004 | 0.2 |
| | Inulin | 2,979 | 0.053 | 1.8 |
| | Galactose | 2,881 | 0.495 | 17.2 |
| | Glucono-1,5-lactone | 2,787 | 0.003 | 0.1 |
| | Methyl-a-D-glucoside | 2,832 | 0.267 | 9.4 |
| | α-cyclodextrin | 2,936 | 0.044 | 1.5 |
| | α-D-(+)-melibiose | 2,687 | 0.197 | 7.3 |
| | Sucrose | 2,798 | 0.058 | 2.1 |
| | Sodium L-aspartate | 2,793 | 0.102 | 3.7 |
| | L-aspartic acid | 2,255 | 0.005 | 0.2 |
| | L-asparagine | 2,739 | 0.044 | 1.6 |
| | Glycine | 2,838 | 0.094 | 3.3 |
| | D-glutamic acid | 2,671 | 0.149 | 5.6 |
| | D-phenylalanine | 2,750 | 0.062 | 2.3 |
| | D-proline | 2,825 | 0.079 | 2.8 |
| | D-a-alanine | 2,818 | 0.09 | 3.2 |
| | DL-isoleucine | 2,770 | 0.047 | 1.7 |
| | L-glutamine | 2,752 | 0.046 | 1.7 |
| | L-(−)-proline | 2,696 | 0.048 | 1.8 |
| | L-arginine | 1,529 | 0.001 | 0.1 |
| | L-serine | 2,598 | 0.078 | 3.0 |
| | L-triptophan | 2,686 | 0.034 | 1.3 |
| | L-valine | 2,747 | 0.043 | 1.6 |
| | L-histidine | 2,733 | 0.015 | 0.5 |
| | L-phenylalanine | 2,650 | 0.031 | 1.2 |
| | L-lysine hydrochloride | 2,694 | 0.093 | 3.5 |
| | L-leucine | 2,733 | 0.038 | 1.4 |
| | Sarcosine | 2,709 | 0.081 | 3.0 |
| | Taurine | 2,513 | 0.081 | 3.2 |

### INDUSTRIAL APPLICABILITY

According to the present invention, by enhancing the stability of the GDH composition, it becomes possible to reduce the thermal deactivation upon production of the glucose measurement reagent, the glucose assay kit and the glucose sensor to reduce the amount of the enzyme to be used and enhance the accuracy of the measurement. It also becomes possible to provide the reagent for measuring the blood glucose level using the GDH composition excellent in storage stability.

1. A method for enhancing stability of glucose dehydrogenase (GDH) comprising a step of making one or more compounds selected from amino acids and sugars which are not substrate of the enzyme coexist with the enzyme in a composition comprising soluble GDH requiring a coenzyme, wherein the stability is enhanced compared with a case where the compound is not made coexist.

2. The method for enhancing the stability according to claim 1 wherein a final concentration of each compound coexisting in a solution is 0.01% by weight or more and a total concentration of respective compounds is 30% by weight or less.

3. The method for enhancing the thermal stability according to claim 1 characterized in that the compounds to be added are one or more, selected from the group consisting of trehalose, mannose, melezitose, sodium gluconate, sodium hi glucuronate, galactose, methyl-a-D-glucoside, cyclodextrin, a-D-melibiose, sucrose, cellobiose, glycine, alanine, serine, BSA, sodium chloride, sodium sulfate, trisodium citrate, ammonium sulfate, succinic acid, malonic acid, glutaric acid, arabinose, sorbitan, 2-deoxy-D-glucose, xylose, fructose, sodium aspartate, glutamic acid, phenylalanine, proline, lysine hydrochloride, sarcosine and taurine.

4. The method for enhancing the thermal stability according to any of claim 1 characterized in that GDH requiring a flavin compound as the coenzyme is derived from filamentous fungus.

5. A composition comprising soluble GDH whose thermal stability has been enhanced by the method according to any of claim 1.

6. The composition containing GDH according to claim 5 characterized in that 20% or more of a GDH activity is left after being treated at 50° C. for 15 minutes compared with the activity in the composition stored at 4° C. in the composition comprising recombinant GDH binding a flavin compound.

7. The composition containing GDH according to claim 5 characterized in that 10% or more of a GDH activity is left after being treated at 50° C. for 30 minutes compared with the activity in the composition stored at 4° C. in the composition comprising flavin compound-binding GDH.

8. A method for measuring a glucose concentration using the composition according to claim 5.

9. A glucose sensor comprising the composition according to any of claim 5.

10. A method for producing a composition comprising a step of making one or more compounds selected from sugars or amino acids which are not substrate of glucose dehydrogenase (GDH) coexist with GDH in a composition comprising soluble, coenzyme-binding GDH, wherein thermal stability of GDH has been enhanced compared with a case where the compound is not made coexist.
